(19) United States
(12) Patent Application Publication
(10) Pub. No.: US 2014/0100448 A1
(43) Pub. Date: Apr. 10, 2014

(54) ROTARY SAMPLE-COLLECTION NEEDLE

(71) Applicant: Cook Medical Technologies LLC, Bloomington, IN (US)

(72) Inventor: John Neilan, Co. Galway (IE)

(73) Assignee: Cook Medical Technologies LLC, Bloomington, IN (US)

(21) Appl. No.: 14/044,263

(22) Filed: Oct. 2, 2013

Related U.S. Application Data
(60) Provisional application No. 61/712,063, filed on Oct. 10, 2012.

(51) Int. Cl. A61B 10/02; A61B 8/08

(52) U.S. Cl. CPC A61B 10/0275; A61B 8/0841
USPC 600/424; 600/566; 600/567

(57) ABSTRACT

A rotary tissue-collection needle configured is provided with a closed distal tip and a distal aperture including a generally longitudinal cutting edge configured to excise tissue into the aperture for collection by rotation of the needle. The needle may be provided as part of a system including an outer sheath within which the needle may be rotated. The needle may be provided with echogenicity-enhancing features.

### CROSS-REFERENCE TO RELATED APPLICATIONS

This application claims priority to U.S. provisional application Ser. No. 61/712,063, filed Oct. 10, 2012, which is incorporated by reference herein in its entirety.

### TECHNICAL FIELD

Presently disclosed embodiments relate generally to endoscopic surgical devices. More particularly, the disclosed embodiments include a sample-collection needle configured for use during minimally-invasive procedures such as endoscopic procedures to collect samples through fine needle aspiration and/or fine needle biopsy.

### BACKGROUND

Fine needle aspiration (FNA) is a diagnostic biopsy procedure used to obtain a sample from a target site in a patient body. A fine needle (e.g., 19-gauge to 25-gauge) is directed to a target site, and suction is applied to the proximal end of a lumen of the needle to aspirate cells through its distal end. The procedure typically is far less invasive than other biopsy techniques, whether performed percutaneously (e.g., to sample a suspected breast tumor or subcutaneous lesion) or endoscopically (e.g., to sample a suspected cholangiocarcinoma via a duodenoscope). Moreover, advances in endoscopic ultrasound (EUS) technology have helped physicians and patients by providing enhanced ability of a physician to visualize a biopsy needle to obtain a sample of material from a target site without requiring an open incision or use of large-bore needles and/or laparoscopic trocars.

Fine needle biopsy (FNB) can obtain a larger sample size (e.g., a larger number of cells in the sample or a “core” comprising intact adjacent cells held together in similar form to their native location) without requiring a larger-gauge needle or requiring multiple passes of the needle to reliably obtain a diagnostically efficacious sample with regard to the number and integrity of the cells in the sample. FIG. 1 is provided as one illustration of a sample-collection needle 100. Both FNA and FNB techniques may include using a needle 100 with a side aperture 130 having a cutting edge 125 that is transverse to the longitudinal needle axis and/or an open needle distal end 110, both of which will contact and cut and/or scrape target tissue to collect cells, tissue, or fragments. Different configurations may be able to collect different sample types (e.g., intact multi-cell samples useful for histology, cells and fragments useful for cytology, etc.).

Thus, current FNA and FNB techniques typically are quite useful for diagnostic evaluation. However, with both techniques, it is often necessary to conduct several passes—reciprocating the needle longitudinally several times to collect sufficient sample material for analysis, and/or even withdrawing the needle entirely then re-directing it to a target region. This can lead to uncertainty about exactly from where a sample (e.g., tissue and/or cells) was taken. It would be advantageous to provide a needle for use in FNA and/or FNB that is configured to collect a sample from a precisely known location rather than an indefinite location or plurality of locations distributed longitudinally along one or more needle paths.

### BRIEF SUMMARY

Embodiments of needles disclosed here address these problems of the current technology and present advantages over existing needles with regard to both structure and methods. In one aspect a rotary sample-collection needle device may include an elongate tubular cannula with a cannula wall defining a cannula lumen, where the cannula lumen extends longitudinally through the cannula. The cannula may include a closed distal end with an aperture through the cannula wall that is open to the cannula lumen. The aperture is disposed proximally adjacent to the closed distal cannula end. The aperture may include a generally longitudinal side edge/lip defined by a portion of the cannula wall, the lip being configured to extend proximally from a distal portion of the needle side aperture and to include a generally longitudinal cutting edge. This cutting edge is configured to excise sample material when the cannula is rotated in a manner that the edge contacts a target site including the sample material. In certain embodiments, a proximal length of the sheath and needle are configured as flexible for operation through a surgical endoscope such as—for example—a gastrointestinal endoscope or the like.

### BRIEF DESCRIPTION OF THE DRAWINGS

In order to assist the understanding of embodiments of the invention, reference will now be made to the appended drawings, which are not necessarily drawn to scale or proportion, and in which like reference numerals generally refer to like elements. The drawings are exemplary only, and should not be construed as limiting the invention.

FIG. 1 shows a sample prior art tissue-sampling needle device;

FIG. 2 shows a rotary tissue-sampling needle device embodiment with a rotary-cutting side aperture;

FIG. 3 shows another rotary tissue-sampling needle device embodiment with a rotary-cutting side aperture; and

FIGS. 4-4A show yet another rotary tissue-sampling needle device embodiment with a rotary-cutting side aperture.

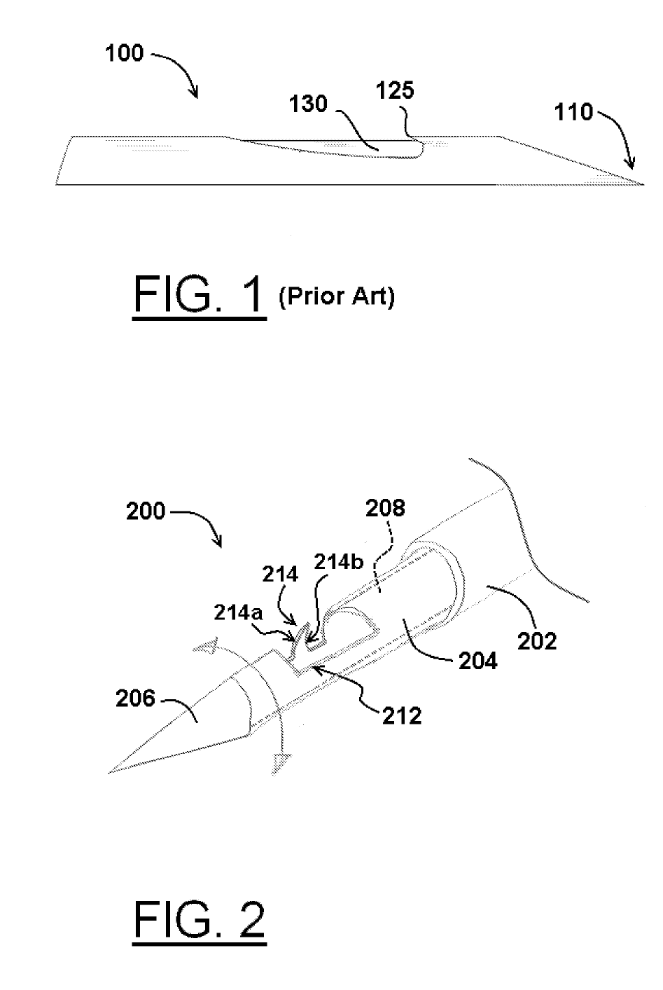

### DETAILED DESCRIPTION

As used herein, the term “proximal” refers to the handle-end of a device held by a user, and the term “distal” refers to the opposite end. The term “surgical visualization device” refers to endoscopes including CCD, ultrasound, fiber optic, and CMOS devices, as well as other devices used for visualizing an internal portion of a patient body such as, for example, a laparoscope or bronchoscope.

One embodiment of a rotary tissue-sampling needle device is described with reference to FIG. 2, which shows the distal end region of a tissue-sampling needle device 200. A proximal handle or hub (not shown) may be constructed for operation in keeping with the present disclosure in a variety of ways that are well-known in the endoscopic needle art. The tissue-sampling needle device 200 includes an elongate outer sheath 202. The sheath 202 includes a longitudinal sheath lumen extending therethrough.

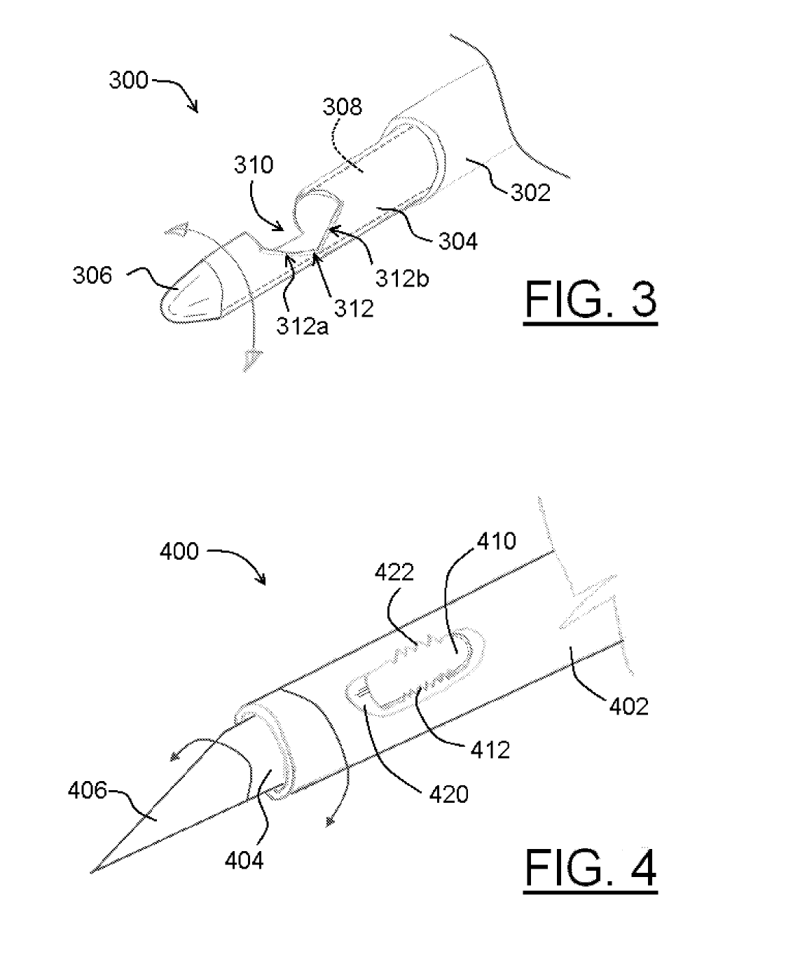

A closed-tip needle 204 extends longitudinally, slidably, and preferably rotatably through the sheath lumen. The closed distal needle tip 206 may be pointed (e.g., conical or some variant thereof, beveled, or otherwise configured in any penetrating-tip manner known or developed in the art). In other embodiments, such as that shown in FIG. 3, it may be rounded or otherwise have a generally atraumatic configuration. A needle lumen 208, configured to communicate a vacuum from the proximal needle end to the distal needle end is provided. A side aperture 210 is open through the needle wall defining the needle lumen 208, near the distal closed tip 206.

The side aperture 210 includes at least one generally longitudinal cutting surface 212 configured to excise sample material upon contact therewith, to capture the material in the needle lumen 208. In the embodiment of FIG. 2, the aperture 210 includes two cutting surfaces that oppose each other across a trans-longitudinal width of the aperture 210. The first cutting surface 212 is generally longitudinally parallel with the central longitudinal axis defined by the needle 204 and the sheath 202. The needle side aperture 210 is generally elongate and its proximal, distal, and first cutting surface edges generally define a Quonset-shaped right-cylindrical section.

However, in this embodiment, a v-shaped cutting edge 214 including two cutting surfaces 214a, 214b, is disposed opposite the first cutting surface 212. The cutting surfaces of this second cutting edge 214 are also generally longitudinal. As used herein, the phrase “generally longitudinal” includes those surfaces and edges disposed parallel with the central longitudinal axis, and also includes surfaces and edges oriented at an angle of or less than about 70 degrees (preferably less than 79 degrees, and more preferably about 60 degrees or less) relative to a plane congruent with the central longitudinal axis. With this structure, in order to excise and capture a sample the needle 204 may be rotated in either direction or both directions around the central longitudinal axis (i.e., the needle being at least generally coaxial with the sheath). This may be most effective when vacuum is exerted to pull sample material into the aperture 210.

Various methods of use will be apparent to those of skill in the art, in view of the present disclosure. In one method, the distal portion of the device 200 may be directed through an endoscope working channel to a location adjacent a target to be sampled. The target may be, for example, a tissue mass (e.g., polyp, tumor) within a patient's body. The needle 204 and/or sheath 202 may be moved in a manner to uncover the needle side aperture 210 within or immediately adjacent the target. Depending upon the location and specific nature of the target, the needle 204 may be used to penetrate within the mass with the sheath still covering the side aperture 210. In other circumstances, the device 200 may be directed immediately adjacent the target, and then the aperture 210 placed in immediate proximity and/or contact with the target. The navigation/ direction to the target may be done using one or more of, for example, the visualization apparatus (e.g., camera or other optics) of an endoscope, ultrasound, and/or fluoroscopic means. A vacuum may be applied through the needle lumen 208 to draw a portion of the target into the aperture 210, whereafter the needle 204 may be rotated in one or both directions around the central longitudinal axis so that the at least one cutting surface (e.g., 212, 214a, and/or 214b) contacts and excises a portion of the target. In various embodiments, the needle 204 may be rotated within the sheath 202 or along with the sheath being rotated. After the sample is excised, the needle 204 may be retracted into the sheath lumen and/or the sheath 202 may be extended to cover the aperture 210.

Another embodiment of a rotary tissue-sampling needle device is described with reference to FIG. 3, which shows the distal end region of a tissue-sampling needle device 300. The tissue-sampling needle device 300 includes an elongate outer sheath 302. The sheath 302 includes a longitudinal sheath lumen extending therethrough.

A closed-tip needle 304 extends longitudinally, slidably, and preferably rotatably through the sheath lumen. The closed distal needle tip 306 is illustrated with a rounded generally atraumatic configuration. This configuration may be useful in certain diagnostic settings where it is desirable not to puncture particular tissue near a target being sampled. A needle lumen 308, configured to communicate a vacuum from the proximal needle end to the distal needle end is provided. A side aperture 310 is open through the needle wall defining the needle lumen 308, near the distal closed tip 306.

The side aperture 310 includes a generally longitudinal cutting surface 312 configured to excise sample material upon contact therewith, to capture the material in the needle lumen 308. It is configured as a V-shaped cutting surface (inverted-v relative to FIG. 2) including two cutting edges 312a, 312b, each of which is generally longitudinally parallel with the central longitudinal axis defined by the needle 304 and the sheath 302. With this structure, in order to excise and capture a sample the needle 304 may be rotated around the central longitudinal axis in the direction faced by the cutting surface 312. The cutting surfaces edge(s) may be beveled or otherwise shaped and/or coated (e.g., with ceramic or other material) to provide a sharp cutting edge that will excise target material.

Depending upon the size of the needle 304 and the particular geometry or other shape of the cutting edge, the sample target material may be collected as multicellular tissue samples suitable for use in histological staining and evaluation, intact cellular samples suitable for cytological evaluation, and/or a mixture of intact and disrupted cells and/or tissues. This may be most effective when vacuum is exerted to pull sample material into the aperture 310, although certain sample types may naturally occupy an immediately-adjacent aperture 310 when it is no longer covered by the outer sheath 302.

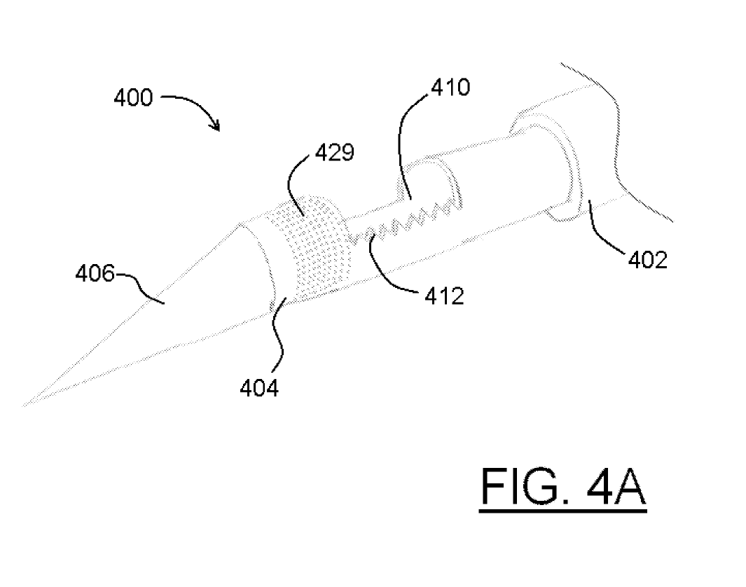

Another embodiment of a rotary tissue-sampling needle device is described with reference to FIG. 4, which shows the distal end region of a tissue-sampling needle device 400. The tissue-sampling needle device 400 includes an elongate outer sheath 402. The sheath 402 includes a longitudinal sheath lumen extending therethrough. The sheath 402 also includes a sheath side-aperture 420.

A closed-tip needle 404 extends longitudinally, slidably, and preferably rotatably through the sheath lumen. The closed distal needle tip 406 is illustrated with a beveled configuration. A needle lumen 408, configured to communicate a vacuum from the proximal needle end to the distal needle end is provided. A side aperture 410 is open through the needle wall defining the needle lumen 408, near the distal closed tip 406.

The side aperture 410 includes a generally longitudinal toothed or serrated cutting surface 412 configured to excise sample material upon contact therewith, to capture the material in the needle lumen 408. The sheath side-aperture 420 includes an opposed cutting surface 422. In the illustrated embodiment, it is also serrated. However, those of skill in the art will appreciate that only one (or neither) of the surfaces need be serrated or otherwise to provide for the excision of target material. The apertures are configured to align in the manner shown, although either may be larger than the other. In this embodiment one or both of the needle 404 and sheath 402 may be rotated relative to the other in order to excise material that has been drawn into the apertures 410, 420 (e.g., by a vacuum applied through the needle lumen). It will generally be preferable to maintain particularly tight tolerances for this embodiment so that target excision may be done via cutting by the sheath cutting edge 420, needle cutting edge 410, and/or by shearing/scissors-like interaction of both cutting edges (one or both of which may be serrated, straight, multi-angled or otherwise configured).

FIG. 4A shows the needle extended from the distal end of the sheath 402 (where the sheath aperture 420 is not shown due to image scale and truncation). In this manner, this device embodiment 400 may alternately be used just like those described above. FIG. 4A also shows echogenic surface features 429 adjacent the needle side aperture 410 and configured for providing ultrasound-visualization of the needle side aperture location. As is known in the art, echogenic surface features may include one or more of dimples, grooves, or other topography on an inner and/or outer surface of the needle cannula 404. These surface features may be used to target and/or identify a specific location from which a sample is being collected in keeping with the goals of the present disclosure.

As noted above, various methods of use for different embodiments will be apparent to those of skill in the art, in view of the present disclosure. In one method, the distal portion of the device 400 may be directed through an endoscope working channel to a location adjacent a target to be sampled. The target may be, for example, a tissue mass (e.g., polyp, tumor) within a patient's body. The needle 404 and/or sheath 402 may be moved in a manner to uncover the needle side aperture 410 within or immediately adjacent the target. This method step may be accomplished by extending the needle 404 out of the sheath lumen or by aligning the needle aperture 410 with the sheath aperture 420. Depending upon the location and specific nature of the target, the needle 404 may be used to penetrate within the mass with the sheath still covering the side aperture 410. In other circumstances, the device 400 may be directed immediately adjacent the target, and then the aperture 410 placed in immediate proximity and/or contact with the target. The navigation/direction to the target may be done using one or more of, for example, the visualization apparatus (e.g., camera or other optics) of an endoscope, ultrasound, and/or fluoroscopic means. A vacuum may be applied through the needle lumen 408 to draw a portion of the target into the aperture(s) 410 (420), whereafter the needle 404 may be rotated in one or both directions around the central longitudinal axis so that the at least one cutting surface (e.g., 412 and/or 422) contacts and excises a portion of the target. In various embodiments, the needle 404 may be rotated within the sheath 402 or along with the sheath being rotated. After the sample is excised, the needle 404 may be retracted into the sheath lumen and/or the sheath 402 may be extended and/or rotated to cover the needle aperture 410. This method and other methods of the present disclosure allow capture of a sample from a specific location without the longitudinal reciprocation commonly required by existing biopsy needles.

The needle device and methods disclosed here provide the advantages associated with FNA and FNB needles of small size (e.g., 19-ga. to 25-ga.) and maneuverability, while offering a means of collecting samples from specifically-identified target sites. These embodiments also are not hampered by the guillotine-style moving parts of other notched needle systems known in the biopsy art (which are generally larger in scale due to a need for having a cutting member that movably transects the notch and/or that may use a cutting-edge on or in conjunction with the overlying sheath in a manner requiring the sheath to shear or “guillotine” the sample).

Those of skill in the art will appreciate that embodiments not expressly illustrated herein may be practiced within the scope of the present invention, including that features described herein for different embodiments may be combined with each other and/or with currently-known or future-developed technologies while remaining within the scope of the claims presented here. For example, the needle cannula, stylet, and side aperture structures disclosed in U.S. Pat. App. Publ. Nos. 2012/0253228 by Schembre et al.; 2013/0006145 by Toomey et al.; and 2013/0006144 by Clancy et al., each of which is incorporated herein by reference in its entirety, may be used or adapted in keeping with the present disclosure within various embodiments while remaining within the scope of the claims presented here. It is therefore intended that the foregoing detailed description be regarded as illustrative rather than limiting. And, it should be understood that the following claims, including all equivalents, are intended to define the spirit and scope of this invention.

1. A rotary sample collection needle device, comprising:
an elongate outer sheath comprising a sheath lumen extending longitudinally therethrough; and
a closed-tip needle extending longitudinally slidably through the sheath lumen, the needle including:
a needle lumen extending longitudinally through the needle, the lumen configured to communicate a vacuum to a distal needle end;
a needle side aperture near the closed tip at the distal needle end, where the side aperture includes at least one generally longitudinal cutting surface configured to excise sample material upon contact therewith, to capture the material in the needle lumen.

2. The device of claim 1, where the at least one generally longitudinal cutting surface is at least partially serrated.

3. The device of claim 1, where the at least one generally longitudinal cutting surface is longitudinally parallel with a central longitudinal axis of the needle.

4. The device of claim 1, where the at least one generally longitudinal cutting surface includes a V-shape.

5. The device of claim 1, where the at least one generally longitudinal cutting surface includes at least two cutting surfaces.

6. The device of claim 1, where the at least one generally longitudinal cutting surface includes at least two cutting surfaces that oppose each other across a trans-longitudinal width of the aperture.

7. The device of claim 6, where at least one of the at least two cutting surfaces includes a serrated edge, a v-shaped edge, or any combination thereof.

8. The device of claim 1, where the needle side aperture is generally elongate and its edges generally define a Quonset-shaped right-cylindrical section.

9. The device of claim 1, where the distal end of the closed tip includes one of a penetratingly pointed or beveled tip or a rounded generally atraumatic tip.

10. The device of claim 1, where the needle further comprises echogenic surface features adjacent the needle side aperture and configured for providing ultrasound-visualization of the aperture location.

11. The device of claim 1, where the elongate outer sheath comprises a sheath side-aperture configured to be selectably alignable with the needle side aperture.

12. The device of claim 11, where the sheath side-aperture includes at least one generally longitudinal sheath cutting-edge configured to align opposably with the at least one generally longitudinal cutting surface of the needle.

13. The device of claim 11, where at least one of the sheath cutting-edge and the at least one cutting edge of the needle is serrated.

14. The device of claim 11, where the needle side aperture includes at least one edge that is generally transverse to a central longitudinal axis of the needle.

15. A sample-collection rotary needle device comprising:
an elongate sheath defining central longitudinal axis and defining a sheath lumen; and
a closed-tip needle disposed longitudinally, slidably, rotatably, and coaxially through the sheath lumen;
where the needle comprises a distal side aperture that includes at least one cutting edge, which cutting edge is
generally parallel with, or at an angle of or less than about 70 degrees relative to, a plane congruent with the central longitudinal axis; and
remains substantially within a region generally defined by an outer circumference of the needle.

16. A method for sample collection in a patient body, the method comprising steps of:
directing a distal portion of the device of claim 1, with the sheath covering the needle side aperture, to a location adjacent a target to be sampled;
moving the needle and/or the sheath so that the needle side aperture is uncovered within or immediately adjacent the target;
providing vacuum through the needle lumen to draw a portion of the target into the aperture; and
rotating the needle so that the at least one cutting surface contacts and excises the portion of the target.

17. The method of claim 16, further comprising a step of moving the needle and/or sheath so that the sheath covers the needle side aperture, enclosing the excised portion of the target.

18. The method of claim 16, where the needle further comprises echogenic surface features adjacent the needle side aperture and the method further comprises ultrasound visualization of the needle relative to the target.

19. The method of claim 16, where the at least one generally longitudinal cutting surface includes at least two cutting surfaces.

20. The method of claim 16,
where the elongate outer sheath comprises a sheath side aperture,
where the sheath side aperture includes at least one generally longitudinal sheath cutting-edge configured to align opposably with the at least one generally longitudinal cutting surface of the needle, and
where the step of rotating the needle further includes the sheath cutting-edge contacting the portion of the target.
